(19) United States
(12) Patent Application Publication
(10) Pub. No.: US 2006/0271174 A1
(43) Pub. Date: Nov. 30, 2006

(54) Mitral Valve Annuloplasty Device with Wide Anchor

(76) Inventors: Gregory Nieminen, Bothell, WA (US); Carly A. Thaler, Seattle, WA (US)

Correspondence Address: WILSON SONSINI GOODRICH & ROSATI, 650 PAGE MILL ROAD, PALO ALTO, CA, 94304-1050, US

(21) Appl. No.: 11/458,042

(22) Filed: Jul. 17, 2006

Related U.S. Application Data
(63) Continuation-in-part of application No. 10/742,585, filed on Dec. 19, 2003.

(51) Int. Cl. A61F 2/24

(52) U.S. Cl. 623/2.36; 606/151; 623/2.11

(57) ABSTRACT

One aspect of the invention provides a tissue shaping device (such as a percutaneous mitral valve annuloplasty device) adapted to be deployed in a vessel to reshape tissue adjacent the vessel. In some embodiments, the tissue shaping device includes a first anchor comprising a flexible wire comprising first and second arms, the first anchor having a deployed configuration in which its width is greater than its height; a second anchor comprising a flexible wire comprising first and second arms; and a connector disposed between the first anchor and the second anchor.

### CROSS-REFERENCE

This application is a continuation-in-part application of Ser. No. 10/742,585, filed Dec. 19, 2003, which is incorporated herein by reference in its entirety and to which application we claim priority under 35 USC § 120.

### BACKGROUND OF THE INVENTION

This invention relates generally to devices and methods for shaping tissue by deploying one or more devices in body lumens adjacent to the tissue. One particular application of the invention relates to a treatment for mitral valve regurgitation through deployment of a tissue shaping device in the patient's coronary sinus or great cardiac vein.

The mitral valve is a portion of the heart that is located between the chambers of the left atrium and the left ventricle. When the left ventricle contracts to pump blood throughout the body, the mitral valve closes to prevent the blood being pumped back into the left atrium. In some patients, whether due to genetic malformation, disease or injury, the mitral valve fails to close properly causing a condition known as regurgitation, whereby blood is pumped into the atrium upon each contraction of the heart muscle. Regurgitation is a serious, often rapidly deteriorating, condition that reduces circulatory efficiency and must be corrected.

Two of the more common techniques for restoring the function of a damaged mitral valve are to surgically replace the valve with a mechanical valve or to suture a flexible ring around the valve to support it. Each of these procedures is highly invasive because access to the heart is obtained through an opening in the patient's chest. Patients with mitral valve regurgitation are often relatively frail thereby increasing the risks associated with such an operation. A device to perform mitral valve annuloplasty is therefore needed that can be implanted percutaneously without opening the chest wall.

### SUMMARY OF THE INVENTION

One aspect of the invention provides a tissue shaping device (such as a percutaneous mitral valve annuloplasty device) adapted to be deployed in a vessel to reshape tissue adjacent the vessel. In some embodiments, the tissue shaping device includes a first anchor comprising a flexible wire comprising first and second arms, the first anchor having a deployed configuration in which its width is greater than its height; a second anchor comprising a flexible wire comprising first and second arms; and a connector disposed between the first anchor and the second anchor. In some embodiments, the first anchor is formed substantially in a figure-8 configuration. In some embodiments, the device includes an anchor lock adapted to lock the first anchor in an expanded configuration and/or an anchor lock adapted to lock the second anchor in an expanded configuration. The first and/or second anchors may be adapted to engage, e.g., a coronary sinus of a heart.

In some embodiments, the device includes a coupler, such as a tether and a hitch wire, adapted to couple the device to a delivery tool. The coupler may be further adapted to release the device from the delivery tool. In some embodiments, the device is adapted to be recaptured by the catheter.

Another aspect of the invention provides a method of performing mitral valve annuloplasty on a patient's heart including the following steps: percutaneously delivering a mitral valve annuloplasty device to a vessel in the patient's heart, the device comprising first and second anchors and a support structure disposed between and operatively connecting the first and the second anchors; expanding the first anchor of the mitral valve annuloplasty device in the vessel to a deployed configuration in which the first anchor's width is greater than its height; and anchoring the second anchor of the mitral valve annuloplasty device. In some embodiments, the delivering step comprises delivering the device to a patient's heart via a catheter.

In some embodiments, the expanding step comprises engaging the first anchor with a coronary sinus and possibly locking the first anchor in the deployed configuration. In some embodiments, the step of anchoring the second anchor comprises expanding the second anchor from a delivery configuration to a deployed configuration in which the second anchor engages a coronary sinus and also possibly locking the second anchor in the deployed configuration.

Some embodiments include the step of capturing the first anchor within a catheter after the first anchoring step, such as by advancing the catheter distally over the first anchor to place the first anchor inside the catheter. Some embodiments include the step of capturing the second anchor within a catheter after the second anchoring step, such as by advancing the catheter distally over the second anchor to place the second anchor inside the catheter.

Some embodiments include the step of applying a proximally directed force on the mitral valve annuloplasty device after the first anchoring step. In some embodiments, the device is uncoupled from a delivery tool after the second anchoring step, such as by releasing a hitch wire from the device and removing a tether from the device.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a superior view of a heart with the atria removed.

FIG. 2 illustrates one embodiment of an intravascular device deployed in a coronary sinus.

FIG. 3 illustrates one embodiment of delivering an intravascular device to a desired location within a patient's body.

FIG. 4 shows one embodiment of an intravascular device with proximal anchor and distal anchor in their expanded and locked configurations.

FIG. 5 shows details of the distal anchor of FIG. 4 with a shaping feature of two entwisted wire segments.

FIG. 6 illustrates an exemplary coupler that may be used with an intravascular device.

FIG. 7 shows an exemplary delivery tool that may be used to deliver and deploy an intravascular device.

### DETAILED DESCRIPTION OF THE INVENTION

The present invention relates to a medical device and uses thereof that supports or changes the shape of tissue near a vessel in which the device is placed. The present invention is particularly useful in reducing mitral valve regurgitation by changing the shape of or supporting a mitral valve annulus. In preferred embodiments, the device comprises a distal anchor adapted to be anchored in the coronary sinus whose width is greater than its height. As used herein, “coronary sinus” refers to not only the coronary sinus itself, but also to the venous system associated with the coronary sinus, including the great cardiac vein.

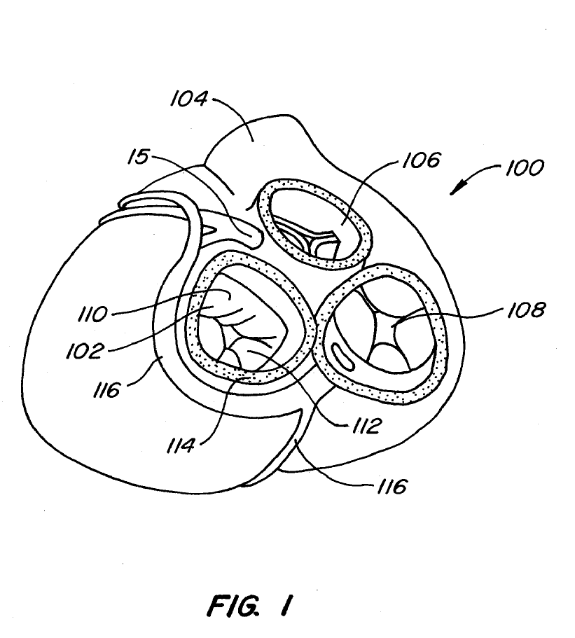

FIG. 1 is a superior view of a heart 100 with the atria removed. As pictured, the heart comprises several valves including mitral valve 102, pulmonary valve 104, aortic valve 106 and tricuspid valve 108. Mitral valve 102 includes anterior cusp 110, posterior cusp 112 and annulus 114. Annulus 114 encircles cusps 110 and 112 and functions to maintain their respective spacing to ensure complete mitral valve closure during left ventricular contractions of the heart 100. As illustrated, coronary sinus 116 partially encircles mitral valve 102 and is adjacent to mitral valve annulus 114. Coronary sinus 116 is part of the venous system of heart 100 and extends along the AV groove between the left atrium and the left ventricle. This places coronary sinus 116 essentially within the same plane as mitral valve annulus 114, making coronary sinus 116 available for placement of shaping device 200 in order to reshape mitral valve geometry and to restore proper valve function.

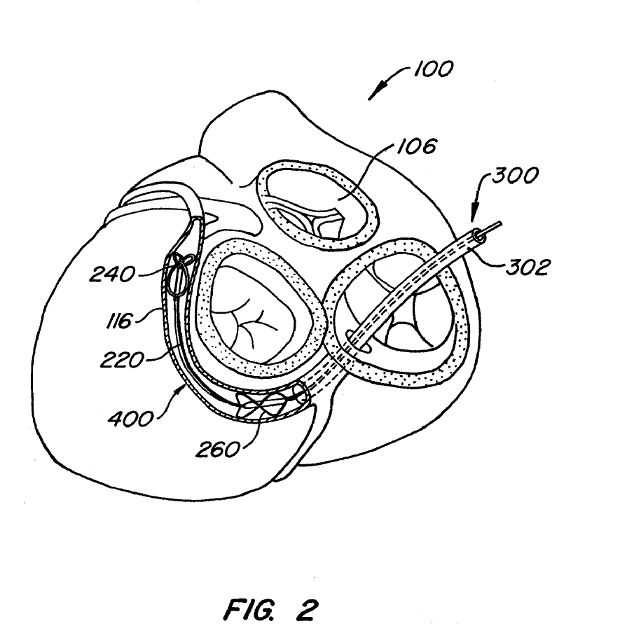

FIG. 2 illustrates one possible embodiment of an intravascular tissue shaping device 400 which is deployable in coronary sinus 116. As illustrated in FIG. 2, device 400 generally comprises an elongated connector 220 disposed between a distal anchor 240 and a proximal anchor 260. Both distal anchor 240 and proximal anchor 260 are shown in their deployed, or expanded, configurations, securely positioned within the coronary sinus 116. FIG. 2 further depicts, in phantom, a delivery tool 300 comprising catheter 302 for delivering and positioning intravascular device 400 in the coronary sinus 116.

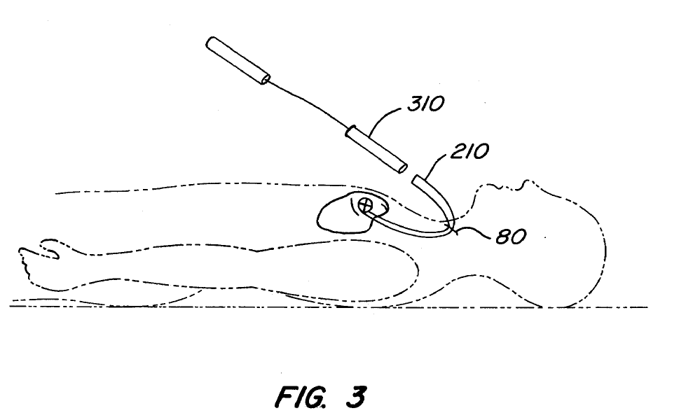

FIG. 3 illustrates one embodiment of delivering the intravascular device of the present invention to a desired location within a patient's body. An incision 80 is made in the patient's skin to gain access to a blood vessel. The blood vessel may be, for example, the jugular vein. A guide catheter 210 is advanced through the patient's vasculature until its distal end is positioned near the desired location for the intravascular device. After positioning the guide catheter 210, a delivery catheter and advancing mechanism 310 are inserted through the guide catheter 210 to deploy the intravascular device at the desired location in the patient's body. In preferred embodiments, the delivery catheter is advanced until its distal end is inside the coronary sinus.

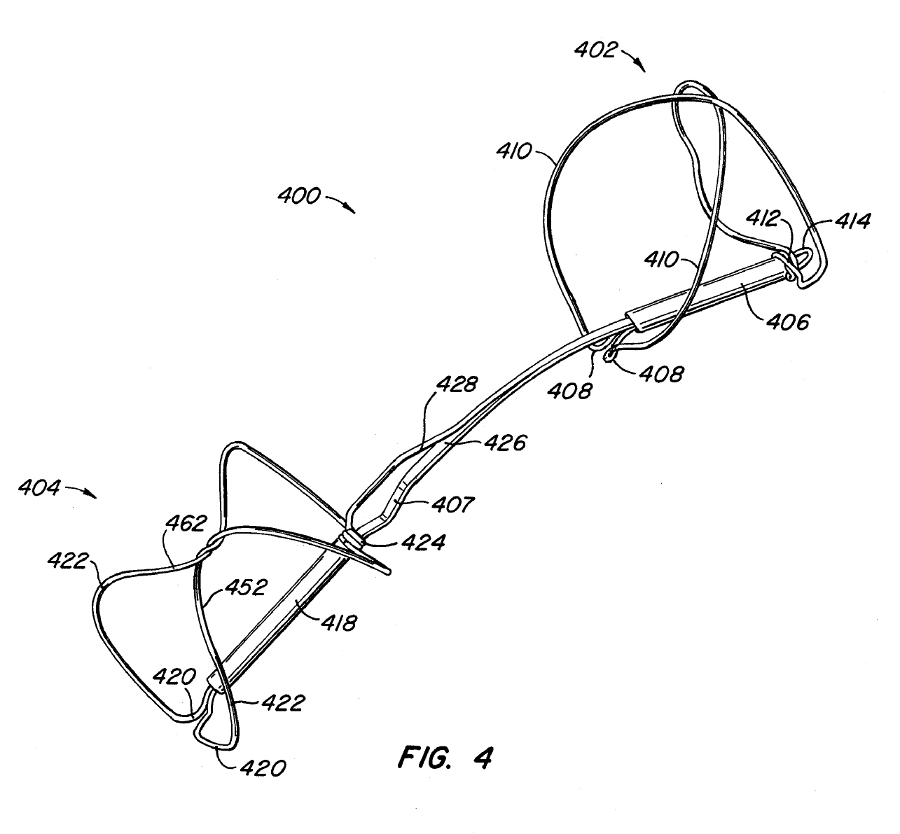

FIG. 4 shows one embodiment of an intravascular shaping device 400 with proximal anchor 402 and distal anchor 404 in their expanded and locked configurations. In this embodiment, proximal anchor 402 is made from a shape memory metal wire, for example Nitinol, extending from a crimp 406. Stress relief portions 408 of the wire extend distal to crimp 406. The wire extends upward from stress relief portions 408 to form vessel engagement portions 410 which cross to form a figure-8 pattern, as shown. Vessel engagement portions 410 and crimp 406 engage the inner wall of the coronary sinus or other vessel in which the device is implanted. The vessel may be a superior vena cava as described in U.S. patent application Ser. No. 11/279,352, filed Apr. 11, 2006. The wire also forms a lock loop 412 which interacts with an arrowhead-shaped element 414 extending from the proximal end of the crimp to form the proximal anchor lock. The proximal side of proximal anchor 402 may be provided with variable slope recapture features, as described in U.S. patent application Ser. No. 10/429,172, filed May 2, 2003.

Likewise, the distal anchor is made from a shape memory wire extending from a crimp 418. Stress relief portions 420 of the wire extend distal to crimp 418. The wire extends upward from stress relief portions 420 to form vessel engagement portions 422 which twist around one another, which is described in further detail below. Vessel engagement portions 422 and crimp 418 engage the inner wall of the coronary sinus or other vessel in which the device is implanted. The wire also forms a lock loop 424. A bent portion 407 of connector 426 interacts with wire portion 428 and lock loop 424 to form a distal anchor lock to secure the distal anchor in an expanded configuration. Actuation of the proximal and distal anchor locks is further described in U.S. patent application Ser. Nos. 10/946,332 and 10/945,855.

Extending between anchors 402 and 404 are a substantially flat connector 426 and a wire connector 428. In this embodiment, connectors 426 and 428 are both made of shape memory metal, such as Nitinol. By spanning the distance between proximal anchor 402 and distal anchor 404, connectors 426 and 428 maintain the reshaping force on the tissue.

Fatigue resistant and stress relief characteristics of the connector 426 and stress relief elements 420 and 408 are described in U.S. patent application Ser. No. 11/275,630, filed Jan. 19, 2006.

Prior to use, tissue shaping devices such as those shown in FIG. 4 may be stored in cartridges or other containers, such as described in U.S. patent application Ser. Nos. 10/946,332 and 10/945,855, then delivered to the coronary sinus or other vessel in a delivery catheter, as shown in FIG. 2.

As shown in FIG. 4, the wire forming the distal anchor 404 has one end positioned within crimp 418. After exiting the distal end of the crimp, a first wire segment 452 of the distal anchor extends distally from the distal crimp, then bends radially outward from the longitudinal axis of the crimp. The wire then bends back proximally and radially inward where it twists around a second wire segment 462 at a coupling point substantially at the anchor's apex. The wire then wraps around the connectors 426 and 428 to form distal lock loop 424 before extending radially outwards and distally where it becomes the second wire segment 462. Finally, the second wire segment 462 bends proximally into the distal end of the distal crimp 418.

As can be seen in FIGS. 1 and 2, the location of the coronary sinus in which the distal anchor may be deployed may be tapered as the diameter along the length of the vessel decreases. Branching vessels may also contribute to a non-uniform diameter of the coronary sinus. Thus, the diameter of the coronary sinus where the distal part of the distal anchor contacts the coronary sinus wall may be narrower than the diameter where the proximal part of the distal anchor contacts the coronary sinus wall. Thus, the vessel wall may exert a larger compressive force on the distal part of the anchor than on the proximal part when the anchor is in a deployed configuration. This compressive force may cause compression of the distal end of the anchor, which can be transferred through the anchor and cause an expansion in a proximal part of the anchor. When such a compression occurs in the distal part, the distal anchor may not be able to anchor properly in the vessel. The distal anchor may compress and deform such that the amount of strain on the vessel is not great enough to allow the distal anchor to remain anchored in place.

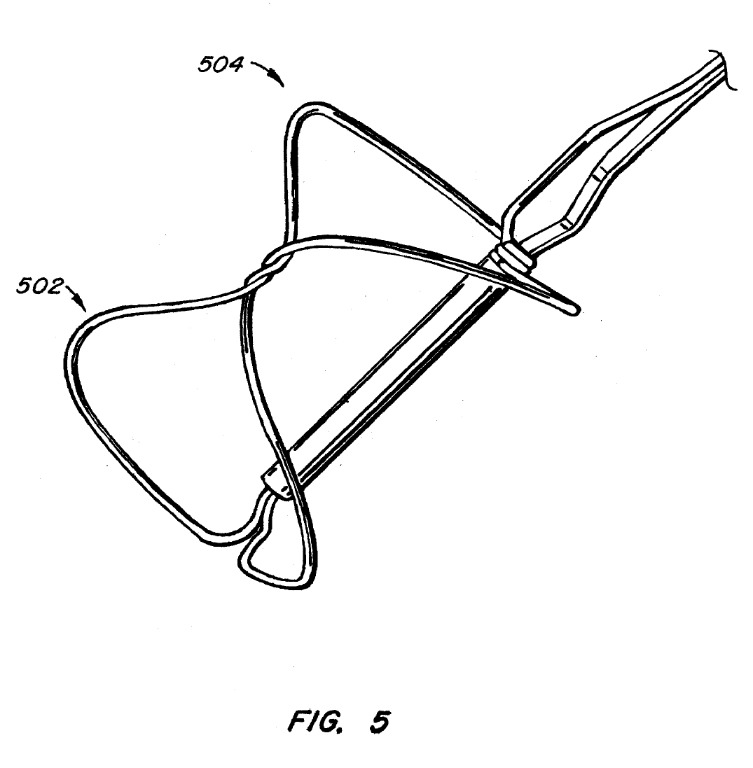

The exemplary embodiment shown in FIGS. 4 and 5 illustrates a device with an anchor shaping feature adapted to resist a compressive force exerted on a distal part of an anchor and resisting an expansion of a proximal part of the anchor in response to a compressive force on the distal part. The device of the present invention resists this compressive force on the distal part of the anchor and allows the device to anchor in place. Particularly, the distal anchor maintains a strain on the vessel which allows for the device to be anchored in the vessel to reduce mitral valve regurgitation, as described below.

As shown in FIG. 5, the distal anchor resists compression of its distal part 502 and resists expansion of its proximal part 504 in response to a compressive force on the distal part 502 of the anchor. Stated alternatively, coupling of the anchor's arms via the twisted wire as shown acts to prevent a compressive force exerted on the distal part of the anchor from being transmitted through the anchor into the proximal part of the anchor. The twist resists a distally exerted compressive force by creating a resistance to such a force.

While the anchor as described thus far resists a compressive force on the distal part of the anchor, the anchor as adapted may also resist a compressive force on the proximal part of the anchor by creating a resistance when a compressive force is exerted on the proximal part of the anchor. Similarly, the proximal anchor of an intravascular device may also be adapted to resist compressive forces from a vessel in which it might be deployed.

While the exemplary embodiments in FIGS. 4 and 5 show one full twist of the two segments of the distal anchor, it is understood that a different number of twists may be used to carry out the intent of the invention and the number of twists shown is not a limitation. In addition, it may be desirable to have a distal anchor with more than two entwisted segments, such as three, four, or more. Furthermore, anchor shaping features other than entwisted wires may be used.

In some embodiments the anchor's width (e.g., the maximum distance between anchor arms 422 in FIG. 4) may be greater than its height (e.g., the distance between crimp 418 and the twisted wire of anchor 404). For example, in some embodiments the distal anchor maybe about 14 mm high and about 17 mm wide. The height and size may, however, vary while still carrying out the purposes of the invention.

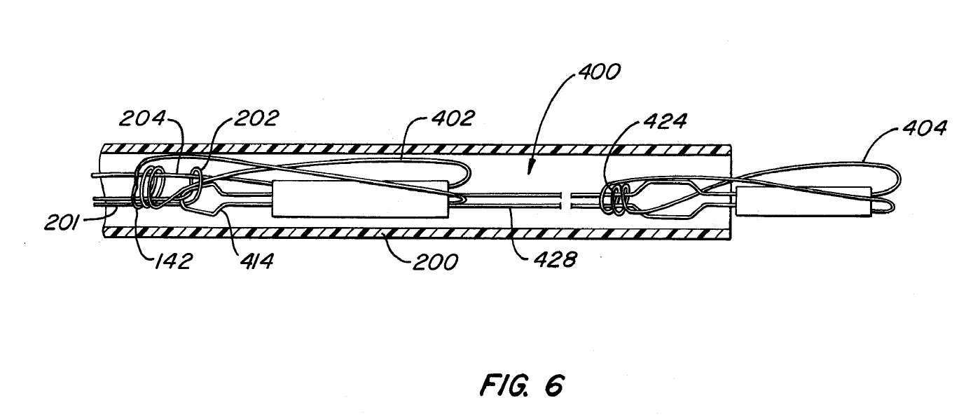

In some embodiments the intravascular device comprises a coupler adapted to couple the intravascular device to a delivery tool. FIG. 6 illustrates an exemplary coupler in use with a different intravascular device that may be used with the intravascular device of this invention. The coupler comprises a loop 202 at the end of tether 201 and a hitch wire 204. Loop 202 extends through arrowhead-shaped element 414, and the hitch wire 204 passes through loop 202 and into the crimp, thereby preventing loop 202 from being withdrawn from arrowhead-shaped element 414.

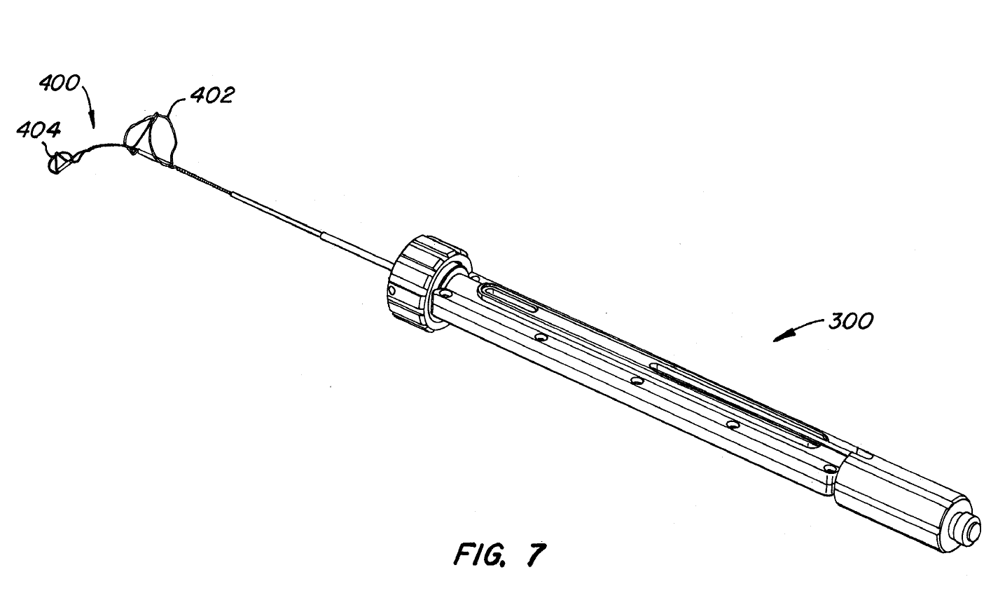

FIG. 7 shows an exemplary delivery tool 300 that may be used to deliver and deploy an intravascular device 400 via a catheter (not shown). Details of the operation of delivery tool 300 may be found in U.S. patent application Ser. Nos. 10/946,332, filed Sep. 20, 2004, and 10/945,855, filed Sep. 20, 2004.

An exemplary method of performing mitral valve annuloplasty on a patient's heart is described. As indicated above, the intravascular device is preferably loaded into and delivered to a desired location within a catheter with the proximal and distal anchors in a delivery or collapsed condition. Medical personnel may deploy the distal end of the intravascular device from the catheter into the lumen of a coronary sinus by advancing the intravascular device or by retracting the catheter, or a combination thereof. A delivery tool such as that of FIG. 7 may provide for distal movement of the intravascular device with respect to the catheter, and a tether may provide proximal movement of the device or for maintaining the position of the intravascular device relative to distal motion of a catheter. Because of the inherent recoverability of the material from which it is formed, the distal anchor begins to expand as soon as it is deployed from the catheter. Using the delivery tool, the distal loop of the distal anchor is moved distally so that the distal anchor further expands and locks in place to securely engage the coronary sinus wall and remains in the locked expanded configuration. The vessel may exert a compressive force on the distal part of the distal anchor, due to, for example, the narrowing diameter of the vessel. The distal anchor as adapted resists compression of the distal part therefore resisting expansion of a proximal part in response to this compressive force. In addition, the greater width of the distal anchor in comparison to its height helps create strain on the vessel to increase the anchoring action.

Next, the intravascular device is tensioned by pulling on the tether to apply a proximally-directed cinching force on the distal anchor, thereby modifying the shape of the coronary sinus and adjacent nearby valve annulus tissue. Fluoroscopy, ultrasound or other imaging technology may be used to detect when the device modifies the shape of the mitral valve annulus sufficiently to reduce mitral valve regurgitation without otherwise adversely affecting the patient. A preferred method of assessing efficacy and safety during a mitral valve procedure is disclosed in co-pending U.S. patent application Ser. No. 10/366,585, filed Feb. 12, 2003. Once the device has been sufficiently cinched, the proximal anchor is deployed from the catheter to begin expansion. In some embodiments, the proximal anchor is deployed in the coronary sinus, but it may be deployed in other vessels as well. The proximal loop of the proximal anchor is advanced distally over the arrowhead-shaped element by the delivery tool to further expand and lock the proximal anchor, thus engaging the coronary sinus wall or other vessel and maintaining a cinching force of the device on the mitral valve annulus. Finally, the coupler that couples the intravascular device to a delivery tool can be released. A hitch wire is first withdrawn (by, for example, a hitch wire actuator of the delivery tool of FIG. 7), thereby releasing the loop so it can be pulled through the proximal lock, and thereby uncoupling the intravascular device from the delivery tool.

In some embodiments it may be necessary to move or remove the intravascular device after deployment by recapturing the device into a catheter. After the distal anchor is deployed and prior to initial deployment of the proximal anchor, the distal anchor may be recaptured into the delivery catheter by holding the intravascular device in place with a the tether while advancing the catheter distally over the distal anchor so that the entire intravascular device is once again inside the catheter. The distally directed force of the catheter collapses the distal anchor to ease recapture into the catheter. In some embodiments the tether may be used to pull the intravascular device proximally while holding the catheter stationary. Either motion, or a combination of motions, may be used to recapture the distal anchor. Similarly, after deploying the second anchor but prior to releasing the coupler as described above herein, the intravascular device may be captured into the delivery catheter by holding the device in place with the tether while advancing a catheter distally first over a proximal anchor, over the support structure, and finally over a distal anchor. The distally directed force of the catheter collapses the anchors such that they can again fit within the catheter. The tether may also be used to pull the device proximally while holding the catheter stationary. If the coupler has been detached from the device prior to capture, the device may be recaptured into the delivery catheter or another catheter by grasping the proximal end of the device with a tether or grasper and by advancing the catheter distally over the device.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A tissue shaping device adapted to be deployed in a vessel to reshape tissue adjacent to the vessel, comprising:
a first anchor comprising a flexible wire comprising first and second arms, the first anchor having a deployed configuration in which its width is greater than its height;
a second anchor comprising a flexible wire comprising first and second arms; and
a connector disposed between the first anchor and the second anchor.

2. The device of claim 1 wherein the first anchor is formed substantially in a figure-8 configuration.

3. The device of claim 1 further comprising an anchor lock adapted to lock the first anchor in an expanded configuration.

4. The device of claim 1 further comprising an anchor lock adapted to lock the second anchor in an expanded configuration.

5. The device of claim 1 further comprising a coupler adapted to couple the device to a delivery tool.

6. The device of claim 5 wherein the coupler comprises a tether and a hitch wire.

7. The device of claim 5 wherein the coupler is further adapted to release the device from the delivery tool.

8. The device of claim 1 wherein the device is adapted to be recaptured by the catheter.

9. The device of claim 1 wherein the first anchor is adapted to engage a coronary sinus.

10. The device of claim 1 wherein the second anchor is adapted to engage a coronary sinus.

11. A method of performing mitral valve annuloplasty on a patient's heart comprising:
percutaneously delivering a mitral valve annuloplasty device to a vessel in the patient's heart, the device comprising first and second anchors and a support structure disposed between and operatively connecting the first and the second anchors;
expanding the first anchor of the mitral valve annuloplasty device in the vessel to a deployed configuration in which the first anchor's width is greater than its height; and
anchoring the second anchor of the mitral valve annuloplasty device.

12. The method of claim 11 wherein the delivering step comprises delivering the device to a patient's heart via a catheter.

13. The method of claim 11 wherein the expanding step comprises engaging the first anchor with a coronary sinus.

14. The method of claim 13 further comprising locking the first anchor in the deployed configuration.

15. The method of claim 13 wherein the step of anchoring the second anchor comprises expanding the second anchor from a delivery configuration to a deployed configuration in which the second anchor engages a coronary sinus.

16. The method of claim 15 further comprising locking the second anchor in the deployed configuration.

17. The method of claim 11 further comprising capturing the first anchor within a catheter after the first anchoring step.

18. The method of claim 17 wherein the capturing step comprises advancing the catheter distally over the first anchor to place the first anchor inside the catheter.

19. The method of claim 11 further comprising capturing the second anchor within a catheter after the second anchoring step.

20. The method of claim 19 wherein the capturing step comprises advancing the catheter distally over the second anchor to place the second anchor inside the catheter.

21. The method of claim 11 further comprising applying a proximally directed force on the mitral valve annuloplasty device after the first anchoring step.

22. The method of claim 11 further comprising uncoupling the device from a delivery tool after the second anchoring step.

23. The method of claim 22 where the uncoupling comprises releasing a hitch wire from the device and removing a tether from the device.
